(19) United States
(12) Patent Application Publication
(10) Pub. No.: US 2021/0177997 A1
(43) Pub. Date: Jun. 17, 2021

(54) MICROSPHERES CONTAINING RADIOACTIVE ISOTOPES AND OTHER MARKERS AND ASSOCIATED METHODS

(71) Applicant: Bard Peripheral Vascular, Inc., Tempe, AZ (US)

(72) Inventors: Christopher D. Drobnik, Wauconda, IL (US); Terry Cruse, Lisle, IL (US)

(73) Assignee: Bard Peripheral Vascular, Inc., Tempe, AZ (US)

(21) Appl. No.: 17/054,618

(22) PCT Filed: May 17, 2019
(86) PCT No.: PCT/US19/32986

§ 371 Date: Nov. 11, 2020

Related U.S. Application Data
(60) Provisional application No. 62/673,632, filed on May 18, 2018.
(60) Provisional application No. 62/673,628, filed on May 18, 2018.

(51) Int. Cl. A61K 51/12; A61K 51/06; A61K 9/50; A61K 49/00

(52) U.S. Cl. CPC A61K 51/1255; A61K 49/0091; A61K 9/5089; A61K 51/06

(57) ABSTRACT

Multiphase microspheres for radioembolization include two-phase microspheres and three-phase microspheres prepared by a microfluidic process. The multiphase microspheres include a primary phase and a first secondary phase surrounded by the primary phase. The primary phase includes a first resin. The first secondary phase includes a second resin and at least one of a radioactive isotope or a compound including at least one radioactive element. Three-phase microspheres additionally include a second secondary phase discrete from the first secondary phase and also surrounded by the primary phase. The second secondary phase may be a gas such as air. The microspheres may be formed by a microfluidic process.

### CROSS-REFERENCES TO RELATED APPLICATIONS

This application claims the benefit of priority to U.S. Provisional App. No. 62/673,632, entitled “RADIOEMBOLIZATION DELIVERY DEVICE” filed May 18, 2018, the disclosure of which is incorporated by reference herein; and to U.S. Provisional App. No. 62/673,628, entitled “DUAL-STAGE SYRINGES WITH LOCKING MECHANISM” filed May 18, 2018, the disclosure of which is incorporated by reference herein.

### TECHNICAL FIELD

This disclosure relates generally to radioembolization microspheres and to methods for preparing the microspheres and, more particularly, to multi-phase microspheres for radioembolization therapy and to microfluidic methods for preparing the microspheres.

### BACKGROUND

Embolization therapy is a minimally invasive surgery performed by interventional radiologists. Typical treatments may include entering the vasculature via a minor incision, such as in the arm or leg, and gaining access to the treatment site by use of guidewires and catheters, optionally aided by imaging techniques such as fluoroscopy. The embolic agent at the treatment site embolizes the vessel, blocking off the flow of blood to tumors downstream from the treatment site and resulting in necrosis and/or shrinkage of the tumors.

Radioactivity may be added to embolization therapies by including a radioactive material in the embolic agent. Transarterial radioembolization, for example, is a transcatheter intra-arterial procedure commonly employed for the treatment of malignant tumors. During this procedure, a microcatheter is navigated into a patient's liver where radioembolizing microspheres loaded with a radioactive compound, such as yttrium-90 (⁹⁰Y), are delivered to the targeted tumors. The microspheres embolize blood vessels that supply the tumors while also delivering radiation to kill tumor cells. Commonly, the microspheres are solid or porous glass or polymeric spheres containing ytttium-89 that is converted by neutron irradiation to yttrium-90 or cation exchange resins onto which yttrium-90 is directly loaded.

Radioembolizing microspheres present numerous challenges in their preparation and handling, particularly where reproducibility and consistent reliability is required in therapeutic methods. For example, radioisotopes such as yttrium-90 can leach out of the microspheres over time, leading to inconsistent radiation dosing at the tumor site. Microspheres are challenging to produce with constant diameters and often require complex sieving processes to sort them by size. Microspheres can be more dense than fluid media such as water, physiological saline, or blood and, therefore, are prone to settling. In general, the production of microspheres may require expensive, specialized equipment and time-consuming processes to satisfy custom requirements from a medical practitioner, all with a constant concern of exposing those preparing the microspheres to harmful radiation.

Therefore, ongoing needs exist for embolic agents and preparation methods that enable greater efficiency and reproducibility in preparation of the embolic agents and that address some or all of the previously identified challenges.

### SUMMARY

According to embodiments, a two-phase microsphere includes a primary phase and a first secondary phase surrounded by the primary phase. The primary phase includes a first resin. The first secondary phase includes a second resin and at least one of a radioactive isotope or a compound including at least one radioactive element. The two-phase microspheres may be formed by a microfluidic process.

According to embodiments, a three-phase microsphere includes a primary phase, a first secondary phase surrounded by the primary phase, and a second secondary phase surrounded by the primary phase and discrete from the first secondary phase. The primary phase includes a first resin. The first secondary phase includes a second resin and at least one of a radioactive isotope or a compound including at least one radioactive element. The second secondary phase includes a gas bubble. The three-phase microspheres may be formed by a microfluidic process.

These and other features, aspects, and advantages of the present invention will become better understood with reference to the following description and the appended claims.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a two-phase microsphere according to embodiments of this disclosure.

FIG. 2 is a three-phase microsphere according to embodiments of this disclosure.

FIG. 3 is a schematic diagram of a microfluidic process for preparing two-phase microspheres according to embodiments of this disclosure.

FIG. 4 is a schematic diagram of a microfluidic process for preparing three-phase microspheres according to embodiments of this disclosure.

### DETAILED DESCRIPTION

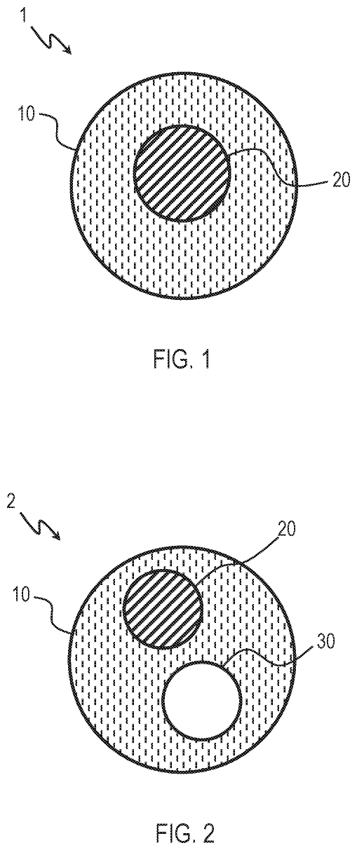

Reference will now be made in detail to embodiments of multiphase microspheres and, particularly, multiphase microspheres for radioembolization. As exemplary embodiments of multiphase microspheres, two-phase microspheres will be described with reference to FIG. 1, and three-phase microspheres will be described with reference to FIG. 2. It should be understood that microspheres having greater than three phases may be prepared as further embodiments by techniques analogous to those for preparing the two-phase microspheres and the three-phase microspheres. Methods for preparing the multiphase microspheres by a microfluidic process will be described subsequently.

Referring to FIG. 1, a two-phase microsphere 1 includes a primary phase 10 and a first secondary phase 20 surrounded by the primary phase 10. The primary phase 10 includes a first resin. The first secondary phase 20 includes a second resin and at least one of a radioactive isotope or a compound including at least one radioactive element. In some embodiments of the two-phase microsphere 1, the primary phase 10, the first secondary phase 20, or both, may further include one or more additional compounds such as a therapeutic agent, a complex of a therapeutic agent, a fluorescent dye, a chelating agent, or a combination of any of these.

Referring to FIG. 2, a three-phase microsphere 2 includes a primary phase 10, a first secondary phase 20 surrounded by the primary phase 10, and a second secondary phase 30 surrounded by the primary phase 10 and discrete from the first secondary phase 20. The primary phase 10 includes a first resin. The first secondary phase 20 includes a second resin and at least one of a radioactive isotope or a compound including at least one radioactive element. The second secondary phase 30 may be chosen from a third resin or a gas. In embodiments for which the second secondary phase 30 is a gas, the gas may be air, nitrogen, or any gas that is generally unreactive with the first resin and the second resin. The second secondary phase 30 may be present within the three-phase microsphere in an amount or volume fraction tailored to render the three-phase microsphere 2 neutrally buoyant in a chosen fluid such as water, physiological saline, blood, or any other carrier fluid suitable for administering embolic microbeads during a radioembolization procedure. In some embodiments of the three-phase microsphere 2, the primary phase 10, the first secondary phase 20, the second secondary phase 30, or any combination thereof, may further include one or more additional compounds such as a therapeutic agent, a complex of a therapeutic agent, a fluorescent dye, a chelating agent, or a combination of any of these.

In both the two-phase microsphere 1 and the three-phase microsphere 2, the primary phase 10 forms the bulk volume of the microsphere that imparts an ability on the microsphere for the microsphere to embolize within a capillary during a radioembolization procedure. The secondary phase 20 of the two-phase microsphere (or the first secondary phase 20 of the three-phase microsphere) has a function of containing or encapsulating the radioactive isotope or compound of the microsphere in a manner that decreases the incidence and likelihood of leaching of radioactive material from the microsphere. In particular, the radioactive isotope or compound remains entrapped within the secondary phase 20, while the primary phase 10 performs the embolic function. In contrast, in a single-phase microsphere, in which a radioactive compound is dispersed in a matrix of a single polymer or resin, some amount of radioactive compound is present out to the outer surface of the microsphere and, therefore, can escape or leach out from the microsphere. Leaching of this nature can lead to unpredictability and unreliability in radiation dosing at a tumor site, for example. The second secondary phase 30 of a three-phase microsphere 2 may be a gas or a third resin. When the second secondary phase 30 of the three-phase microsphere is a gas, such as air, for example, the inclusion of this phase, particularly with respect to a specific volume fraction, can adjust the overall density of the microbead as a whole. As the density of the microbead relates to its buoyancy in a liquid solution such as water, carrier, or blood, the inclusion of a gas phase can optimize flow characteristics of the microbead such as by preventing its settling to the bottom of the liquid solution. When the second secondary phase 30 of the three-phase microsphere is a third resin, the third resin may be selected, for example, to have particular compatibility with additional compounds that may be further included in the microbeads, such as therapeutic agents, for example.

In both the two-phase microsphere 1 and the three-phase microsphere 2, the first resin and the second resin may be identical or different. As used herein, the term “resin” refers to any compound that can exist in an uncured form capable of flowed introduced in the uncured form through a microfluidics system, as will be described subsequently in greater detail, then cured by an additional process such as heating, UV irradiation, or other polymerization reaction, to form a solid, cured material. In some embodiments, the first resin, the second resin, or both, may be insoluble in water and/or physiological fluids such as blood, may be soluble in water and/or physiological fluids such as blood, may be bioresorbable, or may be biodegradable. In some embodiments, the first resin, the second resin, or both, may be water-swellable polymer materials. In some embodiments, the first resin, the second resin, or both, may be stable to gamma irradiation. In some embodiments, the first resin, the second resin, or both, may be impermeable to water. Exemplary compounds suitable as the first resin or the second resin will now be described.

Biodegradable and bioresorbable materials are materials that degrade and/or are reabsorbed safely within the body. Examples of biodegradable and bioresorbable materials may include, without limitation, polyglycolic acid (PGA), polyhydroxy butyrate (PHB), polyhydroxy butyrates-co-beta hydroxyl valerate (PHBV), polycaprolactone (PCL), Nylon-2-nylon-6, polylactic-polyglycolic acid copolymers, PLGA-polyethylene glycol (PEG)-PLGA (PLGA-PEG-PLGA), carboxymethylcellulose-chitosan (CMC-CCN), chitosan, hydroxyethyl acrylate (HEA), iron-based alloys, magnesium-based alloys, and combinations thereof.

Further examples of the first resin, the second resin, or both, include Poly(methylmethacrylate) (PMMA), sulfonated polystyrene-co-divinylbenzene (PSS-DVB), polylactic-co-glycolic acid (PLGA), PLGA-Polyethylene glycol (PEG)-PLGA (PLGA-PEG-PLGA), carboxymethylcellulose-chitosan (CMC-CCN), chitosan, hydroxyethyl acrylate (HEA), poly-4-hydroxybutyrate (P4HB), polyacrylamides and elastin-like proteins, CMC (oxidized carboxymethyl cellulose)/alginate/chitosan, methacrylated hyaluronic acid, cross-linked alginate based polymers (e.g. alginate and poly(N-isopropylacrylamide, alginate-PVA, CMC-alginate,alginate-PCL), functionalized poly(N-isopropylacrylamide), chitosan-alginate, copolymers of N-isopropylacrylamide(NIPAAm)-poly(ethylene glycol)-polycaprotactone-alginate, poly(N-vinylcaprolactam-co-glycidyl methacrylate), poly(N,N-dimethacrylamide-co-glycidyl methacrylate), acrylate based polymers, gelatin-PVA, polyacrylamide gels, polyvinyl alcohols, polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids, and combinations thereof.

In various embodiments, the first resin, the second resin, or both may include a water-swellable polymer material that includes a natural hydrogel polymer such as a chitosan or a polysaccharide, or a synthetic hydrogel polymer such as a polyacrylate, a polyamide, a polyester, a polysaccharide, a poly(methylmethacrylate), or a poly(vinyl alcohol), for example. In some embodiments, the water-swellable polymer material may be biodegradable. Specific examples of water-swellable polymer materials include, without limitation, poly(4-hydroxybutyrate), methacrylated hyaluronic acids (hyaluronic acids being polymers of disaccharides composed of D-glucuronic acid and N-acetyl-D-glucosamine), chitosan-alginates, poly(N-isopropylacrylamide) copolymers, poly(N-isopropylacrylamide)-alginates, poly(N-isopropylacrylamide)-peptides, poly(N-isopropylacrylamide)-α-acryloyloxy-β,β-dimethyl-γ-butyrolactone-hydrophilic Jeffamine, or poly(N-isopropyl-acrylamide)-poly(ethylene glycol) diacrylate-pentaerythritol tetrakis(3-mercapto-propionate). The resins may include may include water-swellable polymer materials that include derivatives of any of the foregoing materials, or may include combinations of any of the foregoing materials or their derivatives.

Examples of biocompatible resins suitable as the first resin, the second resin, or both, include, without limitation, epoxy resins, polyether ether ketone resins, high-density polyethylenes, or combinations thereof.

Further examples of materials suitable as the first resin, the second resin or both, include polyvinyl alcohols, polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polyolefins, polypropylenes, polymethylmethacrylates, polycaprolactones, polyglycolic acids, poly(lactic-co-glycolic) acids (e.g., poly(d-lactic-co-glycolic) acids), polysulfones, polyethersulfones, polycarbonates, nylons, silicones, linear or crosslinked polysilicones, and copolymers or mixtures thereof. In some embodiments, one or both of the resins can be highly water insoluble, high molecular weight polymers. Examples of such a polymer is a high molecular weight polyvinyl alcohol (PVA) that has been acetylized.

Still further examples of materials suitable as the first resin, the second resin, or both, include poly(D,L-lactide), poly(D,L-lactide-co-glycolide), poly(L-lactide-co-glycolide), poly(L-lactide-co-glycolide-co-ε-caprolactone) and poly(D,L-lactide-co-ethylene glycol), poly(L-lactide-co-ethylene glycol), poly(D,L-lactide-bl-glycolide), poly(L-lactide-bl-glycolide), poly(D,L-lactide-bl-ethylene glycol), poly(L-lactide-bl-glycolide), poly(D,L-lactide-bl-lycolide-bl-caprolactone), poly(L-lactide-bl-glycolide-bl-ethylene glycol, and a poly(ester amide).

Still further examples of materials suitable as the first resin, the second resin, or both, include, without limitation, polycaprolactone, poly(L-lactide), poly(D,L-lactide), poly(D,L-lactide-co-PEG) block copolymers, poly(D,L-lactide-co-trimethylene carbonate), polyglycolide, poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, copoly(ether-esters) (e.g. PEO/PLA), polyalkylene oxalates, polyphosphazenes, PHA-PEG, and combinations thereof. The PHA may include poly(α-hydroxyacids), poly(β-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyro sine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

The two-phase microspheres or the three-phase microspheres according to embodiments may have diameters of a size suitable for radiotherapy or radioembolization medical treatment. In some embodiments, for which the microbeads are intended to become stuck in a capillary, without necessarily fully embolizing, so as to deliver therapeutic radiation at a target site, individual microspheres may have diameters of about 10 micrometers (μm) to about 80 μm, or about 20 μm to about 60 μm, or about 30 μm to about 50 μm, for example. In some embodiments, for which the microbeads are intended to be completely embolic (“bland beads”) and include a therapeutic agent, individual microspheres may have diameters of about 30 micrometers (μm) to about 1500 μm. In other embodiments, the microspheres may have diameters of about 30 μm to about 1500 μm, about 30 μm to about 1000 μm, about 30 μm to about 500 μm, about 30 μm to about 100 μm, about 100 μm to about 1500 μm, about 100 μm to about 1000 μm, about 100 μm to about 500 μm, about 500 μm to about 1500 μm, about 500 μm to about 1000 μm, or about 1000 μm to about 1500 μm.

The two-phase microspheres or the three-phase microspheres according to embodiments may include from about 30% by weight to about 70% by weight, or from about 35% by weight to about 65% by weight, or from about 40% to about 60% by weight, or about 45% by weight to about 55% by weight, or about 50% to about 70% by weight resin, based on the total weight of the individual microspheres. In further embodiments, individual microspheres may include from about 30% by weight to about 70% by weight, or from about 35% by weight to about 65% by weight, or from about 40% to about 60% by weight, or about 45% by weight to about 55% by weight, or about 50% to about 70% by weight resin, based on the total weight of the individual microspheres, where the resin is a water-swellable polymer material.

The two-phase microspheres or the three-phase microspheres according to embodiments may be loaded with a therapeutic agent or with a complex of a therapeutic agent and a carrier. Individual drug-loaded microspheres may include one therapeutic agent or a plurality of therapeutic agents.

In embodiments, the therapeutic agent may be a hydrophilic therapeutic agent, a water-soluble therapeutic agent, or a therapeutic agent that has at least some solubility in an aqueous solution. In some embodiments, the therapeutic agent may be a chemotherapeutic agent having at least some efficacy for treating a disease such as cancer. In some embodiments, the therapeutic agent may be a chemotherapeutic agent having at least some efficacy for treating a cancer such as hepatocellular carcinoma, liver cancer, prostate cancer, or breast cancer. The therapeutic agent may have one or more chemical moieties or atomic centers having a positive or negative charge or affinity. Examples of specific therapeutic agents may include, without limitation, doxorubicin, sorafenib, vandetanib, nivolumab, ipilimumab, regorafenib, irinotecan, epirubicin, pirarubicin, 5-fluorouracil, cisplatin, floxuridine, mitomycin C, derivatives of any of the foregoing, prodrugs of any of the foregoing, therapeutically acceptable salts or crystalline forms of any of the foregoing, or combinations of any of the foregoing. Further examples of suitable therapeutic agents include, without limitation, pirarubicin, mitoxantrone, tepotecan, paclitaxel, carboplatin, pemetrexed, penistatin, pertuzumab, trastuzumab, and docetaxel.

In some embodiments, the therapeutic agent may generally surround the microspheres of the microbead material but lack of covalent chemical bonds between the therapeutic agent and the microbead material. Despite lacking covalent chemical bonds, the therapeutic agent and microbead material may have noncovalent intermolecular interactions such as ionic interactions or a van der Waals interaction. In some embodiments, the therapeutic agent of the drug-loaded microbead may generally surround the microbead material and lack covalent chemical bonds to the polymer backbone water-swellable polymer material, yet the therapeutic agent may be chemically bonded to a functional group of the water-swellable polymer material. In some embodiments, the therapeutic agent is not chemically bonded to the water-swellable polymer material at all.

The microspheres may include an amount of therapeutic agent that has a desired therapeutic effect or activity, based on the intended use for the microspheres. The amount of therapeutic agent in the individual drug-loaded microspheres may be adjusted through particular techniques involved during drug loading, such as loading time, loading temperature, or concentration of therapeutic agent in a loading solution, for example. The amount of therapeutic agent in the individual drug-loaded microspheres may be adjusted through synthetic techniques involved for synthesizing the microspheres themselves, such as through adjusting polymer molecular weights, degree of hydrogel crosslinking, polymer density, or polymer porosity of the water-swellable polymer material. For example, when doxorubicin is the therapeutic agent, the amount of drug loading in the drug-loaded microspheres may be adjusted with respect to the number of negative charges in the polymer backbone of the water-swellable polymer material.

In example embodiments, the microspheres may include from about 1% by weight to about 25% by weight, or from about 1% by weight to about 20% by weight, or from about 1% by weight to about 15% by weight, or from about 2% by weight to about 25% by weight, or from about 5% by weight to about 25% by weight, or from about 10% by weight to about 25% by weight therapeutic agent, based on the total weight of the individual microspheres.

In some embodiments, a drug-loaded microbead may include a complex of a carrier and a therapeutic agent. In the complex, the therapeutic agent may be chemically bonded to the carrier or may be associated with the carrier by a non-covalent means such as encapsulation or a van der Waals interaction. In embodiments, the complex may be embedded within the microbead material. In further embodiments, the complex may be embedded within the water-swellable polymer material. When the complex is embedded within the microbead material, the carrier may be chemically bonded to the microbead material while the therapeutic agent is not chemically bonded to the microbead material. Without intent to be bound by theory, it is believed that when the therapeutic agent is bonded or associated with the carrier but is not chemically bonded to the microbead material, the drug-loaded microspheres may be less susceptible to shrinking as a result of replacing water molecules with drug molecules during drug loading. Accordingly, the final size distribution of the drug-loaded microspheres may be controlled more readily by selecting appropriate microbead sizes before the therapeutic agent is loaded.

In embodiments in which the microbeads include a complex of the carrier and the therapeutic agent, the carrier may be any pharmaceutically-acceptable compound that can complex with or encapsulate the therapeutic agent. In some embodiments, the carrier may have charged chemical groups or chemical groups with dipole moments that interact with corresponding chemical groups of the therapeutic agent having an opposite charge or opposite dipole moment. If the carrier is a polymeric material, the carrier may be a different material from the first resin or the second resin. Non-limiting examples of suitable carriers include polysaccharides, liposomes, polymeric micelles, Pluronics, polycaprolactone-b-methoxy-PEG, poly(aspartic acid)-b-PEG, poly(benzyl-L-glutamate)-b-PEG, poly(D,L-lactide)-b-methoxy-PEG, poly(β-benzyl-L-asparate)-b-PEG). Non-limiting examples of polysaccharides include dextrans and dextran sulfates such as dextran sodium sulfate. In one example embodiment, the carrier may include a dextran sodium sulfate having a weight-average molecular weight of from about 40 kDa (kilodalton) to about 500 kDa, or from about 50 kDa to about 300 kDa, or from about 100 kDa to about 300 kDa, or about 100 kDa to about 200 kDa.

In example embodiments, the microspheres may include from about 1% by weight to about 40% by weight, or from about 1% by weight to about 30% by weight, or from about 1% by weight to about 25% by weight, or from about 1% by weight to about 20% by weight, or from about 5% by weight to about 40% by weight, or from about 10% by weight to about 40% by weight, or from about 20% by weight to about 40% by weight carrier, based on the total weight of the individual microbead.

The two-phase microspheres or the three-phase microspheres according to embodiments include a radioactive isotope or a compound including at least one radioactive element. The radioactive isotope or compound may be a radiotherapeutic agent having at least some efficacy for treating a disease such as cancer. In some embodiments, the radioactive isotope or compound may have at least some efficacy for treating a cancer such as hepatocellular carcinoma, cancer that has metastasized to the liver, prostate cancer, or breast cancer. The radioactive isotope or compound may include a radioisotope such as a beta-gamma emitter that or a gamma emitter that emits sufficient gamma radiation to enable imaging. Examples of specific radioactive isotopes include, without limitation, bismuth-213, boron-10, cesium-131, cesium-137, cobalt-60, dysprosium-165, erbium-169, holmium-166, iodine-125, iodine-131, iridium-192, iron-59, lead-212, lutetium-177, molybdenum-99, palladium-103, phosphorus-32, potassium-42, radium-223, rhenium-186, rhenium-188, samarium-153, selenium-75, sodium-24, strontium-89, technetium-99m, thorium-227, xenon-133, ytterbium-169, ytterbium-177, and yttrium-90. Some other examples include actinium-225, astatine-211, bismuth-213, carbon-11, nitrogen-13, oxygen-15, fluorine-18, cobalt-57, copper-64, copper-67, fluorine-18, gallium-67, gallium-68, germanium-68, indium-111, iodine-123, iodine-124, krypton-81m, rubidium-82, strontium-82, and thallium-201. The microspheres according to embodiments herein may include a compound including any of the foregoing radioactive isotopes. In some specific embodiments, the microspheres may include yttrium-90 or a compound including a yttrium-90 atom such as yttrium phosphate (⁹⁰YPO₄), yttrium sulfate (⁹⁰Y₂(SO₄)₃) or (⁸⁹Y⁹⁰Y(SO₄)₃), or yttrium carbonate (⁹⁰Y₂(CO₃)₃) or (⁸⁹Y⁹⁰Y(CO₃)₃).

In example embodiments, the microspheres include water. In example embodiments, the microspheres may have a low water content such as less than 1% by weight, or less than 0.5% by weight, or less than 0.1% by weight, or less than 0.05% (500 ppm) by weight, or less than 0.02% (200 ppm) by weight, or less than 0.01% (100 ppm) by weight, or less than 0.005 (50 ppm) by weight, or less than 0.002% (20 ppm) by weight, or less than 0.001% (10 ppm) by weight water, based on the total weight of the individual microspheres. Without intent to be bound by theory, it is believed that a low water content of the microbead increases the shelf-life and long-term stability of the microbead. Further, it is believed that water contents significantly greater than 1% by weight (such as 2%, 3%, 5%, or 10%, for example) based on the total weight of the microbead, may lead to decomposition or hydrolysis of the therapeutic agent, instability or breaking apart of the water-swellable polymer, or a combination of these, within a few days or even a few hours, such that the microbead cannot be used for embolization procedures, even if the microbead is rehydrated. It is believed that the shelf-life and long-term stability of having water contents significantly greater than 1% by weight are not sufficiently long to ensure viability of the therapeutic agent over the time period from manufacture of the microbead to use of the in an embolization procedure. It is believed that selection of the water-swellable polymer material may correlate with the ability for water to be removed from the microspheres by lyophilization or other drying technique or combination of drying techniques in an amount sufficient to prevent decomposition of the therapeutic agent.

A low water content of the microbead, as previously described, may be attained by drying techniques. In this regard, the microspheres may be dry or nearly dehydrated compositions of the microspheres containing the embedded therapeutic agent or the embedded complex of the therapeutic agent and the carrier. The microspheres may have a powder-like consistency. Accordingly, the microspheres may be made suitable for injection into a subject being treated by rehydrating the microspheres so that the microspheres may be suitable for embolization. Regardless, the microspheres may be provided in such a form that a physician needs to add only an aqueous solution such as water or physiologically buffered saline solution to the microspheres to prepare the microspheres for use in an embolization procedure.

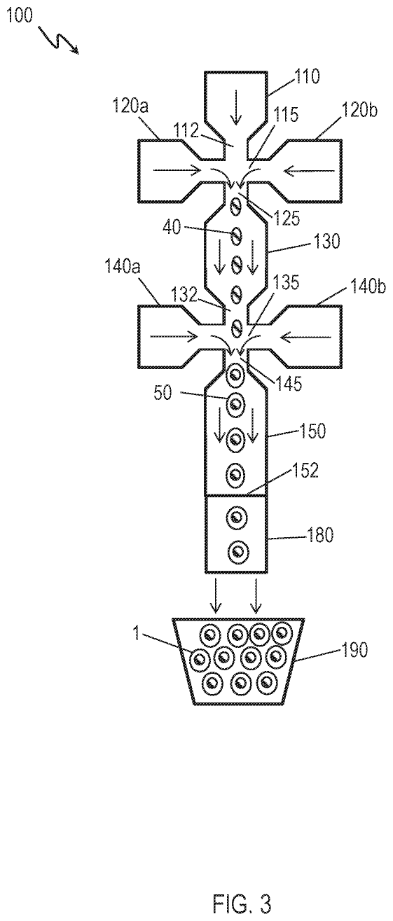

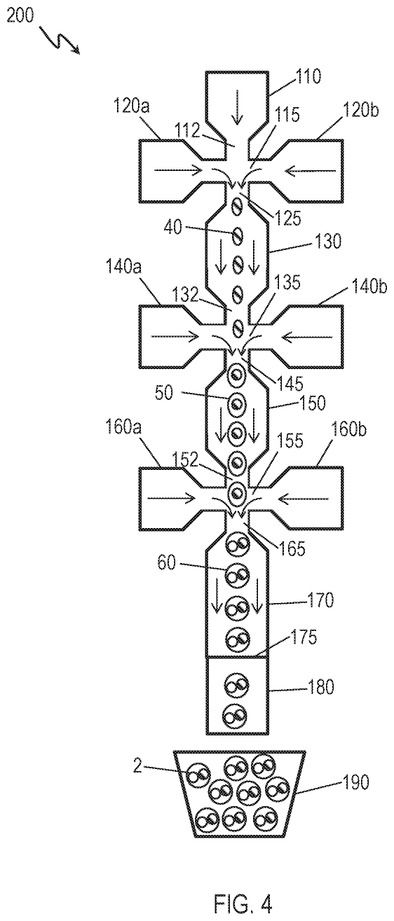

Multiphase microspheres have been described with respect to the embodiments of two-phase microspheres and three-phase microspheres. Methods for preparing the microspheres by microfluidic techniques will now be described with reference to FIGS. 3 and 4.

An example method of preparing two-phase microspheres 1 of FIG. 1 will be described with reference to FIG. 3. The two-phase microspheres 1 may be prepared in a two-tray microfluidic apparatus 100 by flowing a first fluid through a first longitudinal conduit 110 toward a first exit opening 112 of the first longitudinal conduit 110, the first exit opening 112 being in fluidic communication with a first contact zone 115. Simultaneously, a second fluid is flowed through a first transverse conduit that crosses the first longitudinal conduit 110 at the first contact zone 115. In example embodiments, the second fluid may be flowed through the first transverse conduit 120a, 120b from opposing directions toward the first contact zone 115. The first transverse conduit 120a, 120b is in fluidic communication with the first exit opening 112 and with a second exit opening 125 of the first transverse conduit 120a, 120b. The second exit opening 125 is in fluidic communication with a second longitudinal conduit 130. The flow of the first fluid enters the flow of the second fluid in a manner that produces a biphasic stream flowing into the second longitudinal conduit 130 toward a third exit opening 132 of the second longitudinal conduit 130. The third exit opening 132 is in fluidic communication with a second contact zone 135. The biphasic stream is composed of droplets 40 of the first fluid surrounded by a continuous phase of the second fluid.

The example method of preparing two-phase microspheres 1 further includes flowing a third fluid through a second transverse conduit 140a, 140b. The second transverse conduit 140a, 140b crosses the second longitudinal conduit 130 at the second contact zone 135 and is fluidic communication with the third exit opening 132 and with a fourth exit opening 145 of the second transverse conduit 140a, 140b. The fourth exit opening 145 is in fluidic communication with a third longitudinal conduit 150. The flow of the biphasic stream enters the flow of the third fluid in a manner that produces a triphasic stream flowing into the third longitudinal conduit 150 toward a fifth exit opening 152 of the third longitudinal conduit 150. The triphasic stream includes two-phase droplets 50 surrounded by a continuous phase comprising the third fluid. The two-phase droplets 50 have an internal phase comprising the first fluid surrounded by an external phase comprising the second fluid. The triphasic flow including the two-phase droplets 50 then exits through the fifth exit opening 152. The two-phase droplets 50 may be further processed in a processing vessel 180. The processing vessel 180 may include means for curing materials in the two-phase droplets to form cured two-phase microspheres 1 that may be collected in a collecting vessel 190. The processing vessel 180 further may include means for irradiating the two-phase droplets 50 such as by neutron irradiation, for example.

In the exemplary method, the first fluid may include an uncured first resin and at least one of a radioactive isotope or a compound that can be made radioactive upon neutron irradiation of the compound. The uncured first resin may be a compound that, upon curing, polymerizes or reacts to form a first resin, as previously described according to embodiments herein. The uncured first resin may be a compound that, upon curing, polymerizes or reacts to form a first resin that is biresorbable, biodegradable, or both. The uncured first resin may be a compound that, upon curing, polymerizes or reacts to form a first resin that is water-swellable. The radioactive isotope may be an isotope as previously described herein. An example of compounds that can be made radioactive upon neutron irradiation includes yttrium-89 chloride (⁸⁹YCl₃), which may be converted to yttrium-90 chloride (⁹⁰YCl₃) by neutron irradiation.

In the exemplary method, the second fluid includes an uncured second resin. The uncured second resin may be a compound that, upon curing, polymerizes or reacts to form a second resin, as previously described according to embodiments herein. In some embodiments, the second resin is identical to the first resin or is an identical polymer type that differs only in a physical characteristic such as molecular weight, for example. In other embodiments, the second resin is chemically compatible with the first resin but is a different chemical compounds.

The third fluid is a carrier fluid that is immiscible with the second fluid. Example carrier fluids include fluorocarbon oils.

An example method of preparing three-phase microspheres 2 of FIG. 2 will be described with reference to FIG. 4. The three-phase microspheres 2 may be prepared in a three-tray microfluidic apparatus 200 by flowing a first fluid through a first longitudinal conduit 110 toward a first exit opening 112 of the first longitudinal conduit 110, the first exit opening 112 being in fluidic communication with a first contact zone 115. Simultaneously, a second fluid is flowed through a first transverse conduit that crosses the first longitudinal conduit 110 at the first contact zone 115. In example embodiments, the second fluid may be flowed through the first transverse conduit 120a, 120b from opposing directions toward the first contact zone 115. The first transverse conduit 120a, 120b is in fluidic communication with the first exit opening 112 and with a second exit opening 125 of the first transverse conduit 120a, 120b. The second exit opening 125 is in fluidic communication with a second longitudinal conduit 130. The flow of the first fluid enters the flow of the second fluid in a manner that produces a biphasic stream flowing into the second longitudinal conduit 130 toward a third exit opening 132 of the second longitudinal conduit 130. The third exit opening 132 is in fluidic communication with a second contact zone 135. The biphasic stream is composed of droplets 40 of the first fluid surrounded by a continuous phase of the second fluid.

The example method of preparing three-phase microspheres 2 further includes flowing a third fluid through a second transverse conduit 140a, 140b. The second transverse conduit 140a, 140b crosses the second longitudinal conduit 130 at the second contact zone 135 and is fluidic communication with the third exit opening 132 and with a fourth exit opening 145 of the second transverse conduit 140a, 140b. The fourth exit opening 145 is in fluidic communication with a third longitudinal conduit 150. The flow of the biphasic stream enters the flow of the third fluid in a manner that produces a triphasic stream flowing into the third longitudinal conduit 150 toward a fifth exit opening 152 of the third longitudinal conduit 150. The triphasic stream includes two-phase droplets 50 surrounded by a continuous phase comprising the third fluid. The two-phase droplets 50 have an internal phase comprising the first fluid surrounded by an external phase comprising the second fluid. The triphasic flow including the two-phase droplets 50 then exits through the fifth exit opening 152.

The example method of preparing three-phase microspheres 2 further includes flowing a fourth fluid through a third transverse conduit 160a, 160b. The third transverse conduit 160a, 160b crosses the third longitudinal conduit 150 at the third contact zone 155 and is in fluidic communication with the fifth exit opening 150 and with a sixth exit opening 165 of the third transverse conduit 160a, 160b. The sixth exit opening 165 is in fluidic communication with a fourth longitudinal conduit 170. The flow of the biphasic stream enters the flow of the fourth fluid in a manner that produces a tetraphasic stream flowing into the fourth longitudinal conduit 170 toward a seventh exit opening 175 of the fourth longitudinal conduit 170. The tetraphasic stream includes three-phase droplets 60 surrounded by a continuous phase comprising the fourth fluid. The three-phase droplets comprising a first internal phase comprising the first fluid, a second internal phase comprising the second fluid, and an external phase comprising the third fluid. In the three-phase droplets 60, the first internal phase and the second internal phase are discrete from one another and are surrounded by the external phase. Thereupon, the three-phase droplets flow from the seventh exit opening 175 to a processing vessel 180. The processing vessel 180 may include means for curing materials in the three-phase droplets to form cured three-phase microspheres 2 that may be collected in a collecting vessel 190. The processing vessel 180 further may include means for irradiating the three-phase droplets 60 such as by neutron irradiation, for example.

In the exemplary method, the first fluid comprises a gas or an uncured first resin. The uncured first resin may be a compound that, upon curing, polymerizes or reacts to form a first resin, as previously described according to embodiments herein. The gas may be any gas that is unreactive with the first resin or the second resin, such as air or nitrogen, for example. The uncured first resin may be a compound that, upon curing, polymerizes or reacts to form a first resin that is biresorbable, biodegradable, or both. The uncured first resin may be a compound that, upon curing, polymerizes or reacts to form a first resin that is water-swellable. The second fluid comprises an uncured second resin. The uncured second resin may be a compound that, upon curing, polymerizes or reacts to form a second resin, as previously described according to embodiments herein. The uncured second resin may be a compound that, upon curing, polymerizes or reacts to form a second resin that is biresorbable, biodegradable, or both. The uncured second resin may be a compound that, upon curing, polymerizes or reacts to form a second resin that is water-swellable. In some embodiments, the second resin is identical to the first resin or is an identical polymer type that differs only in a physical characteristic such as molecular weight, for example. In other embodiments, the second resin is chemically compatible with the first resin but is a different chemical compounds.

At least one of the first fluid, the second fluid, or both, comprises a radioactive isotope or a compound that can be made radioactive upon neutron irradiation of the compound. The radioactive isotope may be an isotope as previously described herein. An example of compounds that can be made radioactive upon neutron irradiation includes yttrium-89 chloride (⁸⁹YCl₃), which may be converted to yttrium-90 chloride (⁹⁰YCl₃) by neutron irradiation.

The third fluid comprises an uncured third resin, which may be the same as or different from the uncured first resin, the uncured second resin, or both. The fourth fluid is a carrier fluid, such as a fluorocarbon oil, for example, that is immiscible with the third fluid.

In the exemplary methods for preparing either the two-phase microspheres 1 or the three-phase microspheres 2, one or more of the first fluid, the second fluid, the third fluid, or the fourth fluid may further include a therapeutic agent, a complex of a therapeutic agent, a fluorescent dye, a chelating agent, a surfactant, a curing agent, a polymerization inhibitor, or a combination of any of these. Therapeutic agents have been described previously. Fluorescent dyes may include fluorescent compounds, for example, fluorescein. The dye compounds optionally may be derivatized with groups such as acrylic moieties that aid the incorporation of the dye into a resin. Chelating agents generally may ensure retention of the radioactive isotope within the first secondary phase of the multiphase microsphere, or may prevent leaching of the radioactive isotope into the primary phase of the multiphase microsphere. Surfactants, such as polysorbates, for example, stabilize droplets during the microfluidic process. Curing agents, such as ammonium persulfate, for example, enable curing of certain uncured resin materials on exposure thereof to heat. Other curing agents may enable UV curing or radiation curing. Polymerization inhibitors may avoid premature polymerization of the uncured resins during the microfluidic process, as some resins have a tendency to cure from exposure to the radioactivity of the radioactive isotope.

In the exemplary methods for preparing either the two-phase microspheres 1 or the three-phase microspheres 2, the size of the microparticles, the relative amounts of first resin or gas, second resin, third resin, and radioactive isotope or compound may be controlled by adjusting process parameters such as flow rates or pressures of the first fluid, and/or the second fluid, and/or the third fluid, and/or the fourth fluid.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A multiphase microsphere for radioembolization, the multiphase microsphere comprising:
a primary phase comprising a first resin; and
a first secondary phase surrounded by the primary phase, the first secondary phase comprising a second resin and at least one of a radioactive isotope or a compound including at least one radioactive element.

2. The multiphase microsphere of claim 1, wherein the first resin is a bioresorbable resin or a biodegradable resin.

3. The multiphase microsphere of claim 1 or claim 2, wherein the first resin is a water-swellable polymer.

4. The multiphase microsphere of any of the preceding claims, wherein the first resin and the second resin are identical.

5. The multiphase microsphere of any of the preceding claims, wherein the radioactive isotope comprises a beta emitting isotope or a gamma emitting isotope.

6. The multiphase microsphere of any of the preceding claims, wherein the multiphase microsphere further comprises a therapeutic agent.

7. The multiphase microsphere of any of the preceding claims, wherein the primary phase, the secondary phase, or both, further comprises a fluorescent dye.

8. The multiphase microsphere of any of the preceding claims, wherein the first resin is stable to gamma irradiation.

9. The multiphase microsphere of any of the preceding claims, wherein the first resin is impermeable to water.

10. The multiphase microsphere of any of the preceding claims, further comprising a second secondary phase surrounded by the primary phase, the second secondary phase comprising a gas.

11. The multiphase microsphere of claim 10, wherein the gas is air.

12. The multiphase microsphere of claim 10 or 11, wherein the multiphase microsphere is neutrally buoyant in water.

13. The multiphase microsphere of claim 10 or 11, wherein the multiphase microsphere is neutrally buoyant in human blood.

14. A method for forming two-phase microspheres, the method comprising:
flowing a first fluid through a first longitudinal conduit toward a first exit opening of the first longitudinal conduit, the first exit opening being in fluidic communication with a first contact zone;
flowing a second fluid through a first transverse conduit, the first transverse conduit crossing the first longitudinal conduit at the first contact zone and being in fluidic communication with the first exit opening and with a second exit opening of the first transverse conduit, the second exit opening being in fluidic communication with a second longitudinal conduit, whereby a biphasic stream flows into the second longitudinal conduit toward a third exit opening of the second longitudinal conduit, the third exit opening being in fluidic communication with a second contact zone, the biphasic stream comprising droplets of the first fluid surrounded by a continuous phase of the second fluid;
flowing a third fluid through a second transverse conduit, the second transverse conduit crossing the second longitudinal conduit at the second contact zone and being in fluidic communication with the third exit opening and with a fourth exit opening of the second transverse conduit, the fourth exit opening being in fluidic communication with a third longitudinal conduit, whereby a triphasic stream flows into the third longitudinal conduit toward a fifth exit opening of the third longitudinal conduit, the triphasic stream comprising two-phase droplets surrounded by a continuous phase comprising the third fluid, the two-phase droplets comprising an internal phase comprising the first fluid surrounded by an external phase comprising the second fluid; and
flowing the two-phase droplets from the fifth exit opening to a curing vessel; and
curing the two-phase droplets to form two-phase microspheres,
wherein:
the first fluid comprises an uncured first resin and at least one of a radioactive isotope or a compound that can be made radioactive upon neutron irradiation of the compound;
the second fluid comprises an uncured second resin;
the third fluid is immiscible with the second fluid.

15. The method of claim 14, wherein the first resin is a bioresorbable resin or a biodegradable resin.

16. The method of claim 14 or claim 15, wherein the first resin is a water-swellable polymer.

17. The method of any of claims 14 to 16, wherein the first resin and the second resin are identical.

18. The method of any of claims 14 to 17, wherein the radioactive isotope comprises yttrium-90.

19. The method of any of claims 14 to 17, wherein the first fluid comprises the uncured first resin and a compound that can be made radioactive upon neutron irradiation of the compound, the method further comprising neutron irradiating the two-phase microspheres.

20. The method of claim 19, wherein the compound comprises yttrium-89 and the neutron irradiation converts the yttrium-89 to yttrium-90.

21. The method of any of the claims 14 to 20, wherein the first fluid, the second fluid, or both, further comprises a therapeutic agent.

22. The multiphase microsphere of any of the preceding claims, wherein the first fluid, the second fluid, or both, further comprises a fluorescent dye.

23. A method for forming three-phase microspheres, the method comprising:
flowing a first fluid through a first longitudinal conduit toward a first exit opening of the first longitudinal conduit, the first exit opening being in fluidic communication with a first contact zone;
flowing a second fluid through a first transverse conduit, the first transverse conduit crossing the first longitudinal conduit at the first contact zone and being in fluidic communication with the first exit opening and with a second exit opening of the first transverse conduit, the second exit opening being in fluidic communication with a second longitudinal conduit, whereby a biphasic stream flows into the second longitudinal conduit toward a third exit opening of the second longitudinal conduit, the third exit opening being in fluidic communication with a second contact zone, the biphasic stream comprising droplets of the first fluid surrounded by a continuous phase of the second fluid;
flowing a third fluid through a second transverse conduit, the second transverse conduit crossing the second longitudinal conduit at the second contact zone and being in fluidic communication with the third exit opening and with a fourth exit opening of the second transverse conduit, the fourth exit opening being in fluidic communication with a third longitudinal conduit, whereby a triphasic stream flows into the third longitudinal conduit toward a fifth exit opening of the third longitudinal conduit, the fifth exit opening being in fluidic communication with a third contact zone, the triphasic stream comprising two-phase droplets surrounded by a continuous phase comprising the third fluid, the two-phase droplets comprising an internal phase comprising the first fluid surrounded by an external phase comprising the second fluid; and
flowing a fourth fluid through a third transverse conduit, the third transverse conduit crossing the third longitudinal conduit at the third contact zone and being in fluidic communication with the fifth exit opening and with a sixth exit opening of the third transverse conduit, the sixth exit opening being in fluidic communication with a fourth longitudinal conduit, whereby a tetraphasic stream flows into the fourth longitudinal conduit toward a seventh exit opening of the fourth longitudinal conduit, the tetraphasic stream comprising three-phase droplets surrounded by a continuous phase comprising the fourth fluid, the three-phase droplets comprising a first internal phase comprising the first fluid, a second internal phase comprising the second fluid, and an external phase comprising the third fluid, the first internal phase and the second internal phase being surrounded by the external phase; and
flowing the three-phase droplets from the seventh exit opening to a curing vessel; and
curing the three-phase droplets to form three-phase microspheres,
wherein:
the first fluid comprises a gas or an uncured first resin;
the second fluid comprises an uncured second resin;
at least one of the first fluid, the second fluid, or both, comprises a radioactive isotope or a compound that can be made radioactive upon neutron irradiation of the compound;
the third fluid comprises an uncured third resin; and
the fourth fluid is immiscible with the third fluid.

24. The method of claim 23, wherein the first resin is a bioresorbable resin or a biodegradable resin.

25. The method of claim 23 or claim 24, wherein the first resin is a water-swellable polymer.

26. The method of any of claims 23 to 25, wherein the first uncured resin and the second uncured resin are identical.

27. The method of any of claims 23 to 26, wherein the radioactive isotope wherein the radioactive isotope comprises a beta emitting isotope or a gamma emitting isotope.

28. The method of any of claims 23 to 27, wherein the first fluid comprises the uncured first resin and a compound that can be made radioactive upon neutron irradiation of the compound, the method further comprising neutron irradiating the two-phase microspheres.

29. The method of claim 28, wherein the compound comprises yttrium-89 and the neutron irradiation converts the yttrium-89 to yttrium-90.

30. The method of any of the claims 23 to 29, wherein the first fluid, the second fluid, or both, further comprises a therapeutic agent.

31. The method of any of claims 23 to 30, wherein the first fluid, the second fluid, or both, further comprises a fluorescent dye.

32. The method of any of the claims 23 to 31, wherein the first fluid is a gas.

33. The multiphase microsphere of claim 32, wherein the gas is air.
